(19) United States
(12) Patent Application Publication
(10) Pub. No.: US 2015/0329489 A1
(43) Pub. Date: Nov. 19, 2015

(54) SAXAGLIPTIN SALTS

(71) Applicant: RANBAXY LABORATORIES LIMITED, New Delhi, Delhi (IN)

(72) Inventors: Ram Chander ARYAN, New Delhi (IN); Ashwani Kumar SINGH, Firozabad (IN); Amit SHARMA, Panchkula (IN); Ramendra Singh RATHORE, Farrukhabad (IN)

(21) Appl. No.: 14/410,154

(22) PCT Filed: Jul. 2, 2013
(86) PCT No.: PCT/IB2013/055429

§ 371 Date: Dec. 22, 2014

(30) Foreign Application Priority Data
Jul. 2, 2012 (IN) 2016/DEL/2012
Dec. 19, 2012 (IN) 3917/DEL/2012

(51) Int. Cl. C07D 209/52; C07C 51/41; C07C 55/07; C07C 53/10

(52) U.S. Cl. CPC C07D 209/52; C07C 53/10; C07C 51/41; C07C 55/07

(57) ABSTRACT

The present invention provides saxagliptin bisulphate, saxagliptin acetate, saxagliptin oxalate, saxagliptin bicarbonate, and saxagliptin carbonate, their polymorphic forms, processes for their preparation, and pharmaceutical compositions thereof.

### FIELD OF THE INVENTION

The present invention provides saxagliptin bisulphate, saxagliptin acetate, saxagliptin oxalate, saxagliptin bicarbonate, and saxagliptin carbonate, their polymorphic forms, processes for their preparation, and pharmaceutical compositions thereof.

### BACKGROUND OF THE INVENTION

Saxagliptin of Formula A, an orally-active inhibitor of the dipeptidyl peptidase IV enzyme, chemically designated as (1S,3S,5S)-2-[(2S)-2-Amino-2-(3-hydroxytricyclo[3.3.1.1^{3,7}]dec-1-yl)acetyl]-2-azabicyclo[3.1.0]hexane-3-carbonitrile, is indicated as an adjunct to diet and exercise to improve glycemic control in adults with type 2 diabetes mellitus.

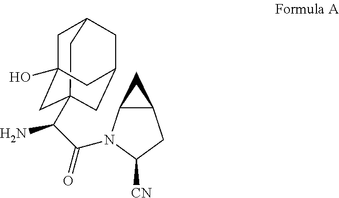 [SMILES: N#C[C@@H]1C[C@@H]2C[C@@H]2N1C(=O)[C@@H](N)C12CC3CC(CC(O)(C3)C1)C2]

U.S. Pat. No. 6,395,767 (hereinafter “the '767 patent”) provides a process for the preparation of the saxagliptin trifluoroacetate salt. The '767 patent also provides the hydrochloride salt of saxagliptin.

U.S. Pat. No. 7,943,656 provides a process for the preparation of crystalline forms of saxagliptin free base, hydrochloride, hydrobromide, hydroiodide, fumarate (2:1), tartrate, benzoate, trifluoroacetate, ammonium sulfate complex, and nitrate.

PCT Publication No. WO 2010/115974 provides a process for the preparation of anhydrous crystalline forms of saxagliptin hydrochloride.

PCT Publication No. WO 2012/017028 provides a process for the preparation of crystalline forms of saxagliptin phosphate.

PCT Publication No. WO 2012/017029 provides a process for the preparation of a crystalline compound comprising a mixture of saxagliptin and an organic C₄-diacid, wherein the organic C₄-diacid is selected from maleic acid, malic acid, L-malic acid, D-malic acid, and succinic acid.

PCT Publication No. WO 2012/047871 provides a process for the preparation of crystalline Form K, Form Z, Form D, and amorphous saxagliptin hydrochloride.

Chinese Publication No. CN 102086172 provides a process for the preparation of mesylate, maleate, malate, succinate, and citrate salts of saxagliptin.

Several processes are known in the literature for making saxagliptin, or a salt thereof, for example, PCT Publication Nos. WO 2004/052850, WO 2005/115982, WO 2005/106011 and WO 2005/094323.

In the pharmaceutical industry there is a constant need to identify the critical physicochemical parameters such as novel salts and novel polymorphic forms that affect the drug's performance, stability, etc., which may play a key role in determining a drug's market acceptance and success.

Since saxagliptin or its salts constitute an important therapeutic agent, additional saxagliptin salts are of value to pharmaceutical science. Thus, there is a need for the development of novel saxagliptin salts having improved solubility, stability, bioavailability, storage and handling stability, and less susceptibility to degradation at lower temperatures.

Accordingly, the present inventors have prepared saxagliptin bisulphate, saxagliptin acetate, saxagliptin oxalate, saxagliptin bicarbonate, and saxagliptin carbonate salts. The salts of the present invention are easy to prepare and isolate in solid forms, particularly, in crystalline forms. Further, they can be prepared by an efficient, economical, and reproducible process, which is particularly suited to large scale preparation.

### SUMMARY OF THE INVENTION

A first aspect of the present invention provides saxagliptin bisulphate.

A second aspect of the present invention provides a crystalline form of saxagliptin bisulphate.

A third aspect of the present invention provides saxagliptin acetate.

A fourth aspect of the present invention provides a crystalline form of saxagliptin acetate.

A fifth aspect of the present invention provides saxagliptin oxalate.

A sixth aspect of the present invention provides a crystalline form of saxagliptin oxalate.

A seventh aspect of the present invention provides a process for the preparation of a compound of Formula I

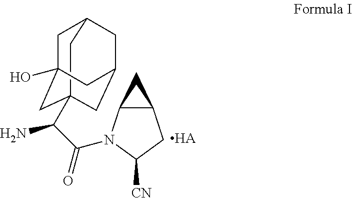

which comprises treating saxagliptin or its salt with HA, wherein HA is selected from sulphuric acid, acetic acid, and oxalic acid.

An eighth aspect of the present invention provides saxagliptin bicarbonate of Formula II.

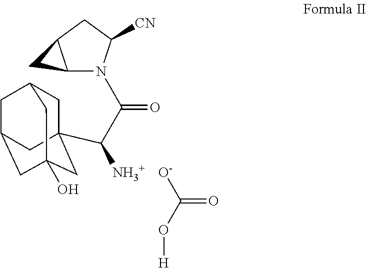

A ninth aspect of the present invention provides a crystalline form of saxagliptin bicarbonate.

A tenth aspect of the present invention provides a process for the preparation of saxagliptin bicarbonate, which comprises contacting saxagliptin or its salt with a suitable carbonate source.

An eleventh aspect of the present invention provides saxagliptin carbonate of Formula III.

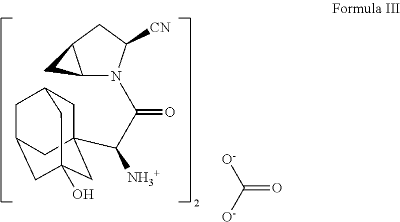

A twelfth aspect of the present invention provides a crystalline form of saxagliptin carbonate.

A thirteenth aspect of the present invention provides a process for the preparation of saxagliptin carbonate, which comprises heating saxagliptin bicarbonate, optionally in the presence of water.

A fourteenth aspect of the present invention provides the use of saxagliptin salts selected from saxagliptin bisulphate, saxagliptin acetate, saxagliptin oxalate, saxagliptin bicarbonate, or saxagliptin carbonate for the preparation of saxagliptin or salts, solvates, or polymorphs thereof.

A fifteenth aspect of the present invention provides a pharmaceutical composition comprising saxagliptin salts selected from saxagliptin bisulphate, saxagliptin acetate, saxagliptin oxalate, saxagliptin bicarbonate, or saxagliptin carbonate, and a pharmaceutically acceptable carrier.

A sixteenth aspect of the present invention provides a method of treating type 2 diabetes mellitus which comprises administering to a patient in need thereof a therapeutically effective amount of saxagliptin salts selected from saxagliptin bisulphate, saxagliptin acetate, saxagliptin oxalate, saxagliptin bicarbonate, or saxagliptin carbonate, and a pharmaceutically acceptable carrier.

### DETAILED DESCRIPTION OF THE INVENTION

A first aspect of the present invention provides saxagliptin bisulphate. A second aspect of the present invention provides a crystalline form of saxagliptin bisulphate. The crystalline form of saxagliptin bisulphate of the present invention may be characterized by an XRPD pattern substantially the same as depicted in FIG. 1, exhibiting interplanar spacing (d) values at about 3.63, 3.39, 3.27, and 3.20 (Å), and further exhibiting interplanar spacing (d) values at about 4.22, 3.89, 3.09, 3.05, 2.98, 2.91, 2.79, and 2.63 (Å). The crystalline form of saxagliptin bisulphate has an XRPD pattern with characteristic peak values (2θ) at about 24.50, 26.26, 27.25, and 27.84±0.2°, and additional characteristic peak values (2θ) at about 21.04, 22.82, 28.89, 29.26, 29.98, 30.71, 32.05, and 34.06±0.2°. The crystalline form of saxagliptin bisulphate of the present invention may be characterized by FTIR as depicted in FIG. 2. The crystalline form of saxagliptin bisulphate of the present invention may be characterized by DSC as depicted in FIG. 3, with a characteristic endothermic peak value at about 100.90° C. in the DSC thermogram.

A third aspect of the present invention provides saxagliptin acetate.

A fourth aspect of the present invention provides a crystalline form of saxagliptin acetate. The crystalline form of saxagliptin acetate of the present invention may be characterized by an XRPD pattern substantially the same as depicted in FIG. 4, exhibiting interplanar spacing (d) values at about 11.97, 9.84, 5.98, 4.96, and 4.91 (Å), and further exhibiting interplanar spacing (d) values substantially at about 6.27, 5.16, 4.71, 4.68, 4.50, 4.45, 3.99, and 3.87 (Å). The crystalline form of saxagliptin acetate has an XRPD pattern with characteristic peak values (2θ) at about 7.38, 8.98, 14.79, 17.85, and 18.03±0.2°, and additional characteristic peak values (2θ) at about 14.12, 17.17, 18.82, 18.92, 19.70, 19.94, 22.26, and 22.97±0.2°. The crystalline form of saxagliptin acetate of the present invention may be characterized by FTIR as depicted in FIG. 5. The crystalline form of saxagliptin acetate of the present invention may be characterized by DSC as depicted in FIG. 6, with characteristic endothermic peak values at about 68.12, 79.62, 152.38, and 161.14° C. in the DSC thermogram.

A fifth aspect of the present invention provides saxagliptin oxalate.

A sixth aspect of the present invention provides a crystalline form of saxagliptin oxalate. The crystalline form of saxagliptin oxalate of the present invention may be characterized by an XRPD pattern substantially the same as depicted in FIG. 7, exhibiting interplanar spacing (d) values substantially at about 12.99, 6.28, 4.96, 4.91, and 4.67 (Å), and further exhibiting interplanar spacing (d) values at about 5.99, 5.93, 5.69, 4.59, 4.32, 4.01, and 3.08 (Å). The crystalline form of saxagliptin oxalate has an XRPD pattern with characteristic peak values (2θ) at about 6.80, 14.06, 17.88, 18.06, and 18.98±0.2°, and additional characteristic peak values (2θ) at about 14.76, 14.93, 15.56, 19.30, 20.54, 22.15, and 28.97±0.2°. The crystalline form of saxagliptin oxalate of the present invention may be characterized by FTIR as depicted in FIG. 8. The crystalline form of saxagliptin oxalate of the present invention may be characterized by DSC as depicted in FIG. 9, with a characteristic endothermic peak value at about 61.67° C., and a characteristic exothermic peak value at about 186.24° C. in the DSC thermogram.

A seventh aspect of the present invention provides a process for the preparation of a compound of Formula I

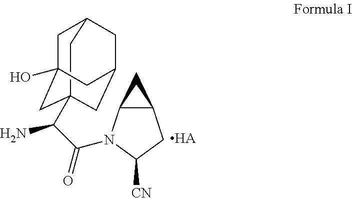

which comprises treating saxagliptin or its salt with HA, wherein HA is selected from sulphuric acid, acetic acid, or oxalic acid.

The saxagliptin or its salt used as the starting material may be prepared by any of the methods known in the art including those described in, for example, U.S. Pat. Nos. 6,395,767, and 7,943,656; PCT Publications WO 2004/052850, WO 2005/115982, WO 2005/106011, WO 2005/094323, WO 2010/115974, WO 2012/017028, WO 2012/017029, and WO 2012/047871.

The saxagliptin or its salt prepared by any of the methods known in the art may be isolated or directly treated with HA.

The saxagliptin or its salt prepared by any of the methods known in the art may be optionally purified prior to treatment with HA to remove foreign particulate matter. Alternatively, it may be treated with activated charcoal to remove coloring and other related impurities in a suitable solvent. The solution of saxagliptin or its salt may be optionally concentrated to reduce the amount of solvent. The saxagliptin salt may optionally be converted to saxagliptin base before treatment with HA.

Treating saxagliptin or its salt with HA may include adding, dissolving, slurrying, stirring, or a combination thereof. Saxagliptin or its salt may be treated with HA directly, or in the presence of a suitable solvent at a suitable temperature.

The term “solvent” includes any solvent, or a solvent mixture, including for example, water, esters, alkanols, halogenated hydrocarbons, ketones, ethers, polar aprotic solvents, or mixtures thereof.

Examples of esters include ethyl acetate, n-propyl acetate, isopropyl acetate, and n-butyl acetate. Examples of alkanols include those primary, secondary, and tertiary alcohols having from one to six carbon atoms. Examples of suitable alkanols include methanol, ethanol, n-propanol, isopropanol, and butanol. Examples of halogenated hydrocarbons include dichloromethane, chloroform, and 1,2-dichloroethane. Examples of ketones include acetone and methyl ethyl ketone. Examples of ethers include diethyl ether and tetrahydrofuran. Examples of polar aprotic solvents include N,N-dimethylformamide, N,N-dimethylacetamide, dimethylsulphoxide, acetonitrile, and N-methylpyrrolidone.

Saxagliptin or its salt is treated with HA at a temperature of from about −10° C. to about 10° C., preferably, from about −5° C. to about 5° C. The formation of the saxagliptin salt may be accelerated by stirring the reaction mixture for about 10 minutes to about 4 hours at a temperature of from about −5° C. to about 40° C., preferably from about 0° C. to about 25° C. After completion of the reaction, the saxagliptin salt of Formula I may be isolated by filtration, decantation, solvent precipitation, trituration, evaporation, distillation, or combinations thereof.

An eighth aspect of the present invention provides a saxagliptin bicarbonate of Formula II.

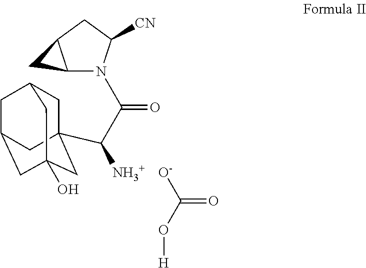

A ninth aspect of the present invention provides a crystalline form of saxagliptin bicarbonate. The crystalline form of saxagliptin bicarbonate of the present invention may be characterized by an XRPD pattern substantially the same as depicted in FIG. 10, exhibiting interplanar spacing (d) values at about 3.70, 2.99, and 2.86 (Å), and further exhibiting interplanar spacing (d) values at about 7.45, 3.17, 3.12, 2.64, 2.39, and 2.30 (Å). The crystalline form of saxagliptin bicarbonate has an XRPD pattern with characteristic peak values (2θ) at about 24.05, 29.84, and 31.18±0.2°, and additional characteristic peak values (2θ) at about 11.86, 28.13, 28.60, 33.88, 37.55, and 39.01±0.2°. The crystalline form of saxagliptin bicarbonate of the present invention may be characterized by FTIR as depicted in FIG. 11. The crystalline form of saxagliptin bicarbonate of the present invention may be characterized by DSC as depicted in FIG. 12, with a characteristic endothermic peak value at about 76.94° C. in the DSC thermogram.

A tenth aspect of the present invention provides a process for the preparation of saxagliptin bicarbonate, which comprises contacting saxagliptin or its salt with a suitable carbonate source.

The saxagliptin or its salt prepared by any of the methods known in the art may be isolated or directly treated with a suitable carbonate source.

The saxagliptin or its salt prepared by any of the methods known in the art, before treatment with a suitable carbonate source, may be optionally purified to remove foreign particulate matter or treated with activated charcoal to remove coloring and other related impurities in a suitable solvent. The solution of saxagliptin or its salt may be optionally concentrated to reduce the amount of solvent. The saxagliptin salt may optionally be converted to saxagliptin base before treatment with a suitable carbonate source, optionally in the presence of a base.

The term “carbonate source” includes carbon dioxide gas, dry ice, and carbonic acid prepared in situ by dissolving carbon dioxide gas in water.

The term “base” includes hydroxides, carbonates, and bicarbonates of alkali and alkaline earth metals; ammonia; alkyl amines; hydrazine; and the like. Examples of hydroxides, carbonates, and bicarbonates of alkali and alkaline earth metals may include lithium hydroxide, sodium hydroxide, potassium hydroxide, sodium carbonate, potassium carbonate, sodium bicarbonate, or potassium bicarbonate. Examples of alkyl amines may include diethyl amine, triethyl amine, or methyl diethyl amine.

Treating saxagliptin or its salt with a suitable carbonate source may include adding, dissolving, slurrying, stirring, or combinations thereof. Saxagliptin or its salt may be treated with a suitable carbonate source directly or in the presence of a suitable solvent at a suitable temperature.

The term “solvent” includes any solvent, or a solvent mixture, including for example, water, alkanols, esters, ketones, polar aprotic solvents, or mixtures thereof. Examples of alkanols include primary, secondary and tertiary alcohols having from one to six carbon atoms. Examples of suitable alkanols include methanol, ethanol, n-propanol, isopropanol, and butanol. Examples of esters include ethyl acetate, n-propyl acetate, isopropyl acetate, and n-butyl acetate. Examples of ketones include acetone and methyl ethyl ketone. Examples of polar aprotic solvents include N,N-dimethylformamide, N,N-dimethylacetamide, dimethylsulphoxide, acetonitrile, and N-methylpyrrolidone.

In a specific embodiment of this aspect, saxagliptin is treated with dry ice at a temperature of from about 20° C. to about 30° C., in ethanol while stirring for about 30 minutes to about 1 hour, preferably, from about 35 minutes to about 45 minutes. Ethanol is removed by distillation under vacuum, followed by the addition of ethyl acetate while stirring. Saxagliptin bicarbonate of Formula II may be isolated by filtration, decantation, solvent precipitation, trituration, evaporation, distillation, or combinations thereof.

An eleventh aspect of the present invention provides saxagliptin carbonate of Formula III.

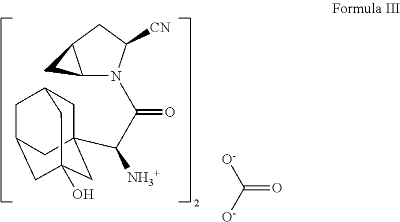

A twelfth aspect of the present invention provides a crystalline form of saxagliptin carbonate. The crystalline form of saxagliptin carbonate of the present invention may be characterized by FTIR as depicted in FIG. 13. The crystalline form of saxagliptin carbonate of the present invention may be characterized by DSC as depicted in FIG. 14. The crystalline form of saxagliptin carbonate has a characteristic endothermic peak value at about 113.45° C. in the DSC thermogram.

A thirteenth aspect of the present invention provides a process for the preparation of saxagliptin carbonate, which comprises heating saxagliptin bicarbonate, optionally in the presence of water.

In a specific embodiment of this aspect, saxagliptin bicarbonate is dissolved in water and stirred at a temperature of from about 50° C. to about 100° C., preferably, from about 65° C. to about 70° C., for about 3 hours to about 6 hours, preferably, for about 4 hours to about 5 hours. Water is then removed by distillation under vacuum. The residue obtained is stirred with ethyl acetate and then removed by distillation under vacuum to obtain the solid. The solid may be washed with ethyl acetate, and is then isolated by filtration, decantation, solvent precipitation, trituration, evaporation, distillation, or combinations thereof to obtain the saxagliptin carbonate.

A fourteenth aspect of the present invention provides the use of saxagliptin salts selected from saxagliptin bisulphate, saxagliptin acetate, saxagliptin oxalate, saxagliptin bicarbonate, or saxagliptin carbonate for the preparation of saxagliptin or salts, solvates, or polymorphs thereof.

Saxagliptin salts may be used for the preparation of saxagliptin by contacting with a base or heating, optionally in the presence of water. The base may be selected from the group comprising of hydroxides, carbonates, and bicarbonates of alkali and alkaline earth metals; ammonia; alkyl amines; hydrazine; and the like. Examples of hydroxides, carbonates, and bicarbonates of alkali and alkaline earth metals include lithium hydroxide, sodium hydroxide, potassium hydroxide, sodium carbonate, potassium carbonate, sodium bicarbonate, or potassium bicarbonate. Examples of alkyl amines may include diethyl amine, triethyl amine, or methyl diethyl amine. Saxagliptin thus obtained may be converted to salts, solvates, or polymorphs thereof.

A fifteenth aspect of the present invention provides a pharmaceutical composition comprising saxagliptin salts selected from saxagliptin bisulphate, saxagliptin acetate, saxagliptin oxalate, saxagliptin bicarbonate, or saxagliptin carbonate, and a pharmaceutically acceptable carrier.

A sixteenth aspect of the present invention provides a method of treating type 2 diabetes mellitus which comprises administering to a patient in need thereof a therapeutically effective amount of saxagliptin salts selected from saxagliptin bisulphate, saxagliptin acetate, saxagliptin oxalate, saxagliptin bicarbonate, or saxagliptin carbonate, and a pharmaceutically acceptable carrier.

### BRIEF DESCRIPTION OF THE FIGURES

FIG. 1 and FIG. 1a depict the X-Ray Powder Diffractogram (XRPD) of saxagliptin bisulphate and the associated values, respectively, prepared as per Example 1.

FIG. 2 depicts the Fourier-Transform Infra-red (FTIR) spectrum of saxagliptin bisulphate prepared as per Example 1.

FIG. 3 depicts the Differential Scanning Calorimetry (DSC) of saxagliptin bisulphate prepared as per Example 1.

FIG. 4 and FIG. 4a depict the XRPD of saxagliptin acetate and the associated values, respectively, prepared as per Example 2.

FIG. 5 depicts the FTIR spectrum of saxagliptin acetate prepared as per Example 2.

FIG. 6 depicts the DSC of saxagliptin acetate prepared as per Example 2.

FIG. 7 and FIG. 7a depict the XRPD of saxagliptin oxalate and the associated values, respectively, prepared as per Example 4.

FIG. 8 depicts the FTIR spectrum of saxagliptin oxalate prepared as per Example 4.

FIG. 9 depicts the DSC of saxagliptin oxalate prepared as per Example 4.

FIG. 10 and FIG. 10a depict the XRPD of saxagliptin bicarbonate and the associated values, respectively, prepared as per Example 5.

FIG. 11 depicts the FTIR spectrum of saxagliptin bicarbonate prepared as per Example 5.

FIG. 12 depicts the DSC of saxagliptin bicarbonate prepared as per Example 5.

FIG. 13 depicts the FTIR spectrum of saxagliptin carbonate prepared as per Example 6.

FIG. 14 depicts the DSC of saxagliptin carbonate prepared as per Example 6.

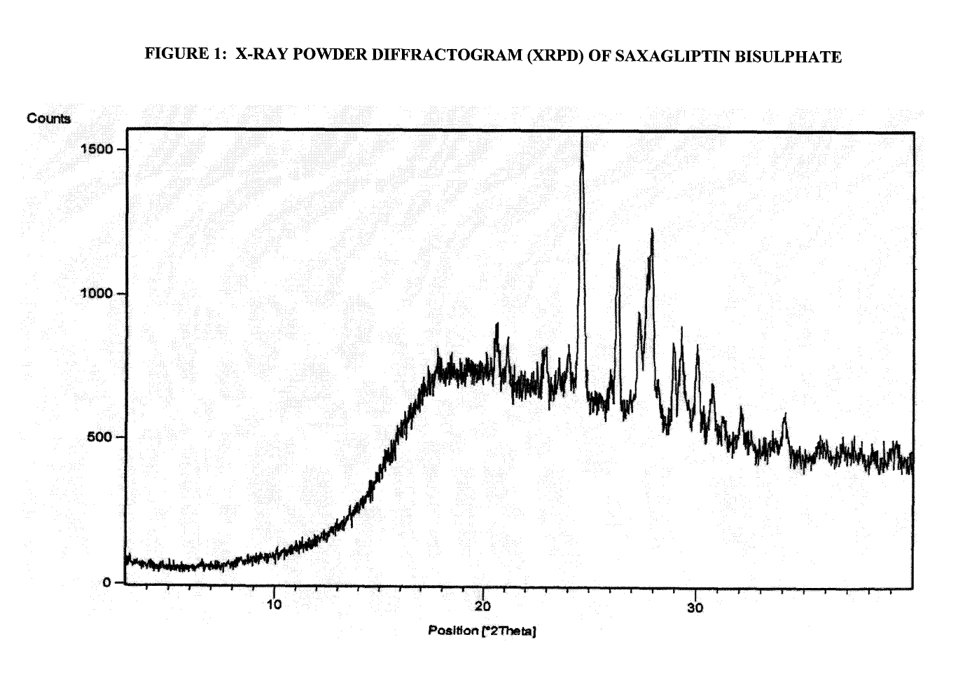

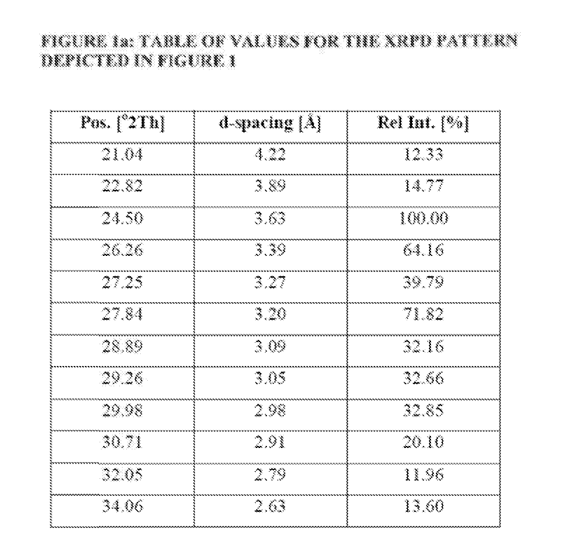

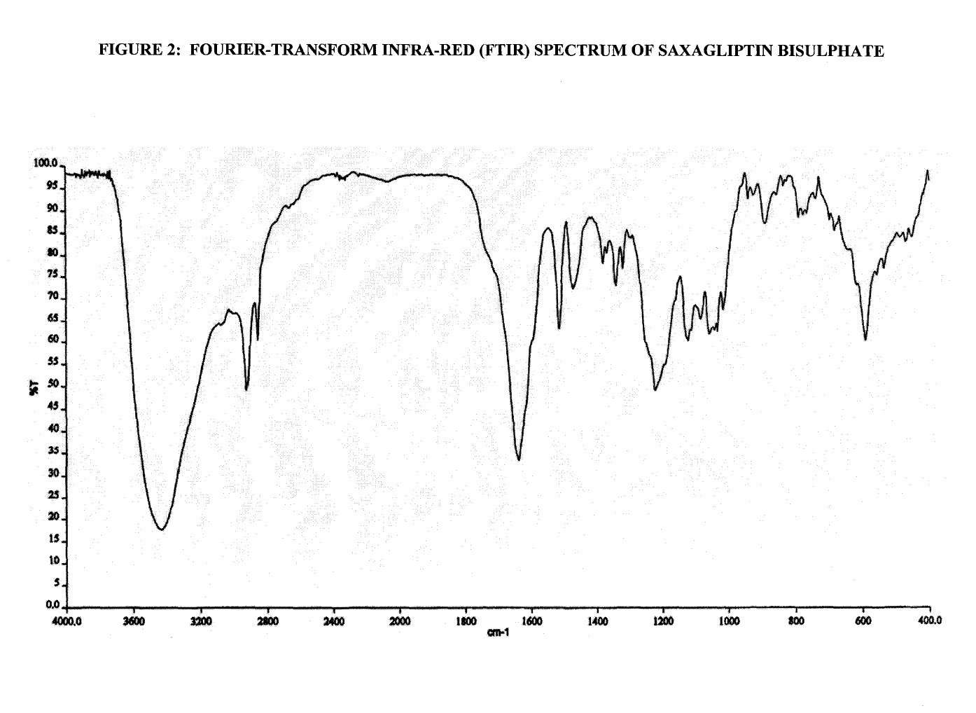

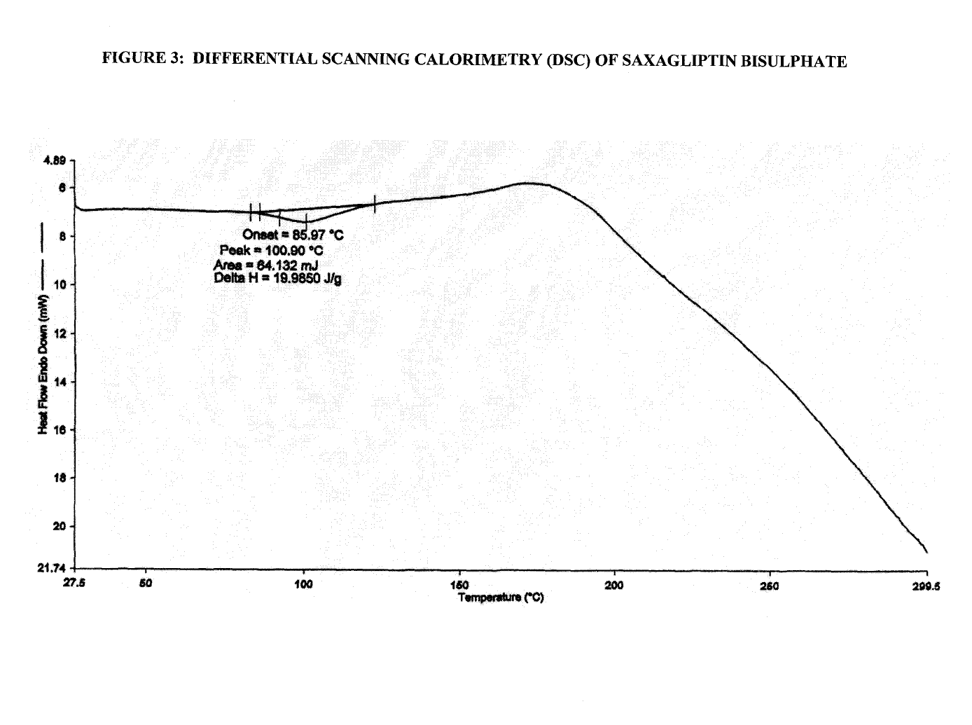

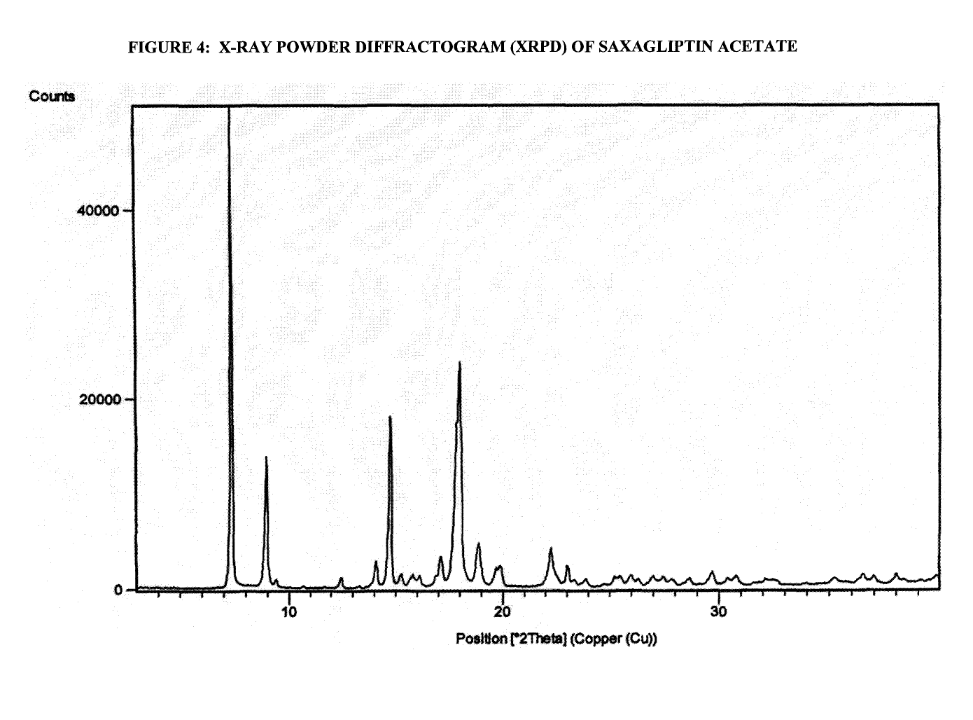

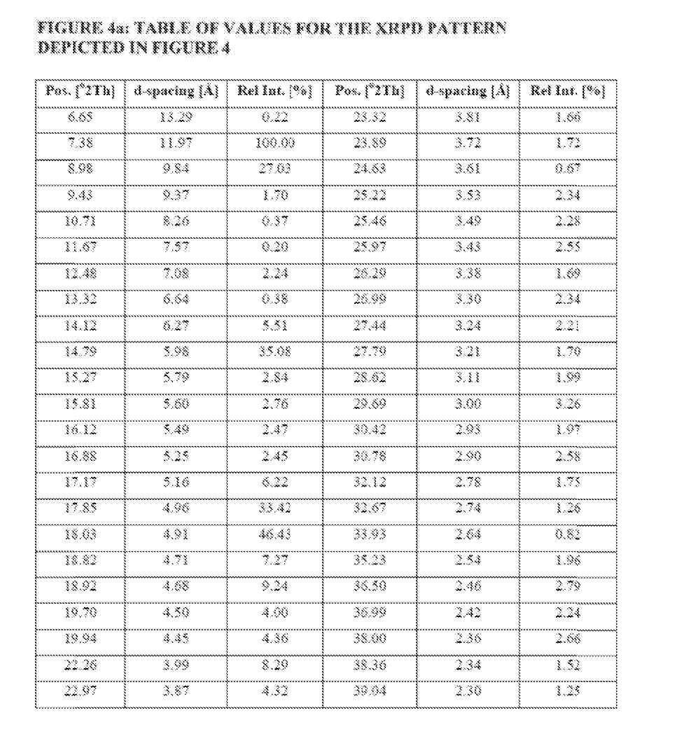

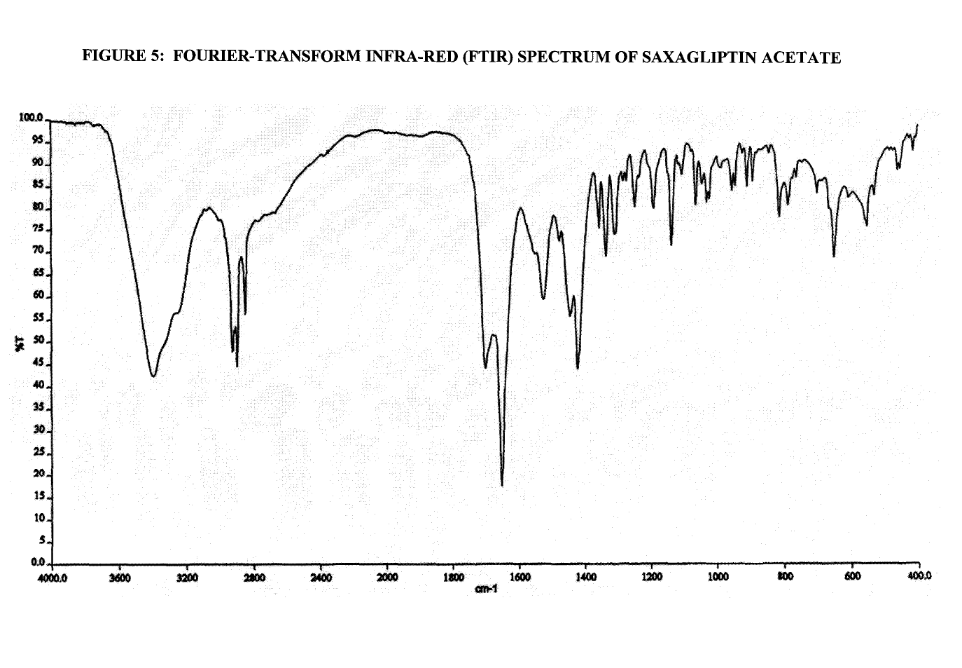

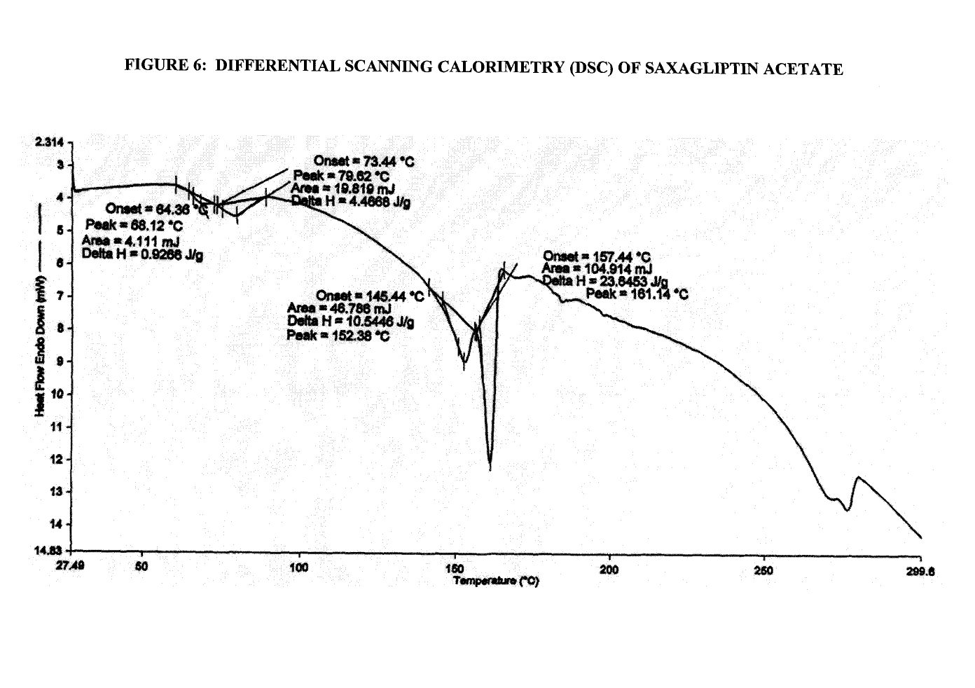

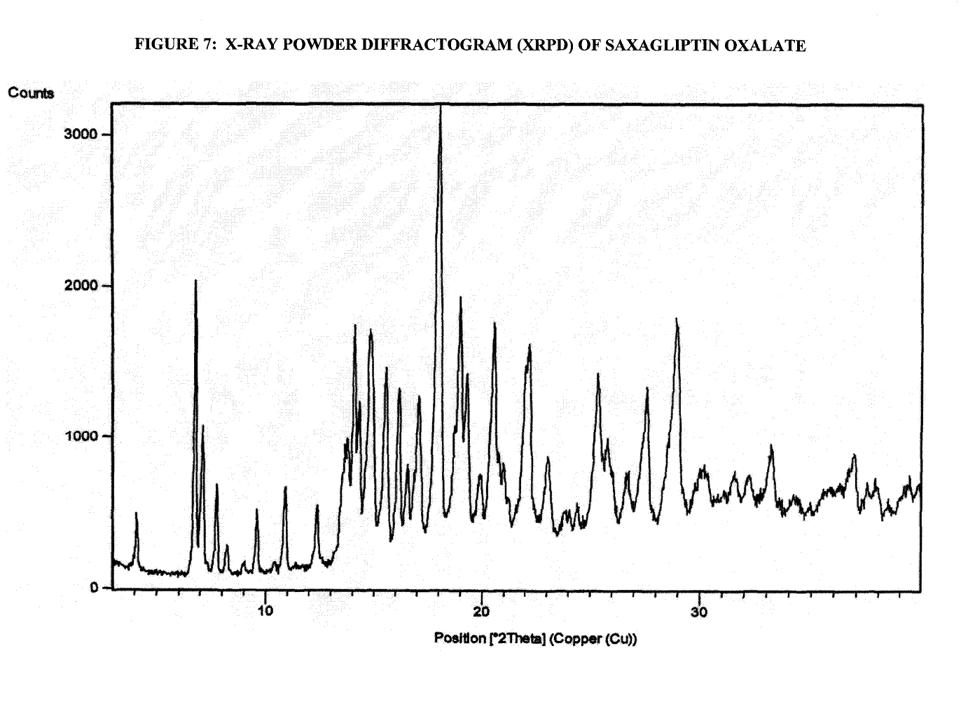

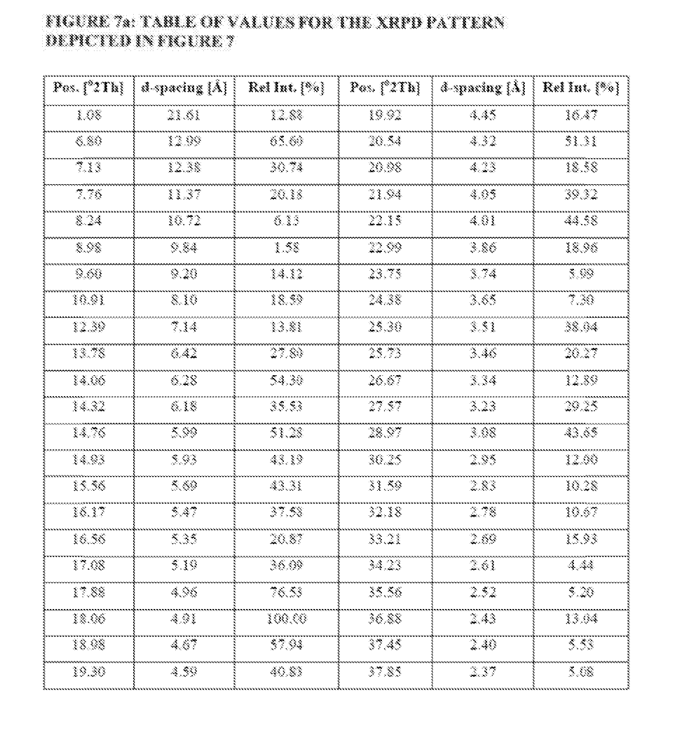

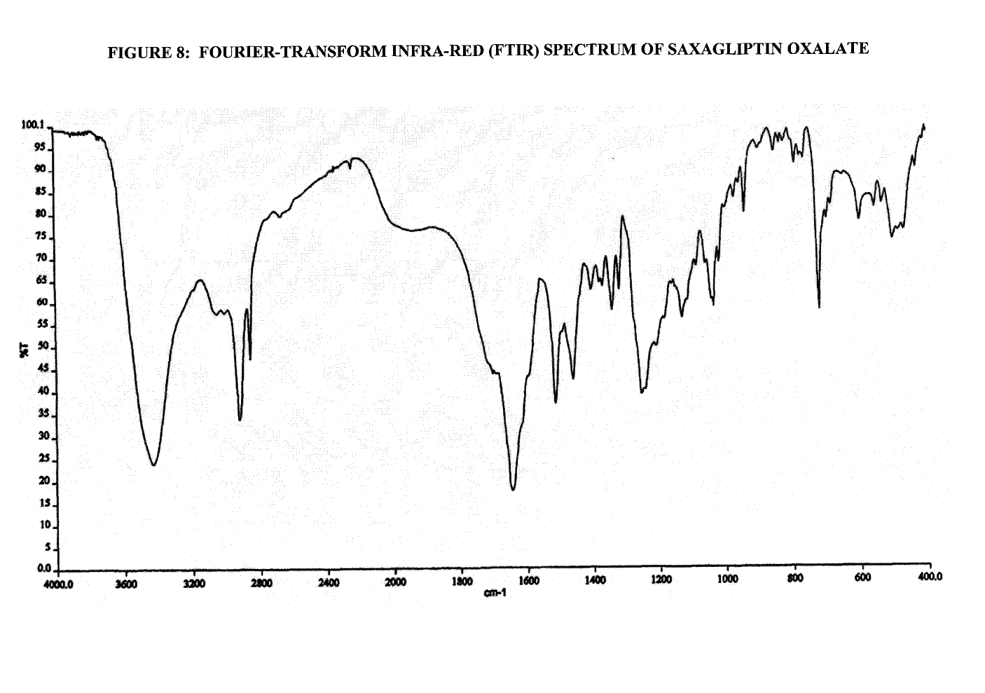

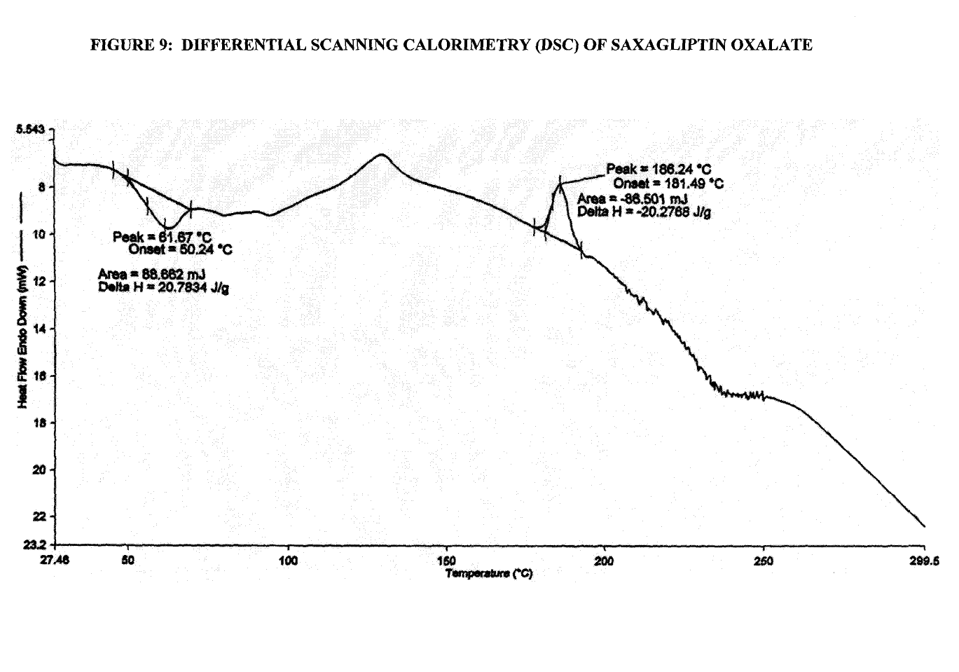

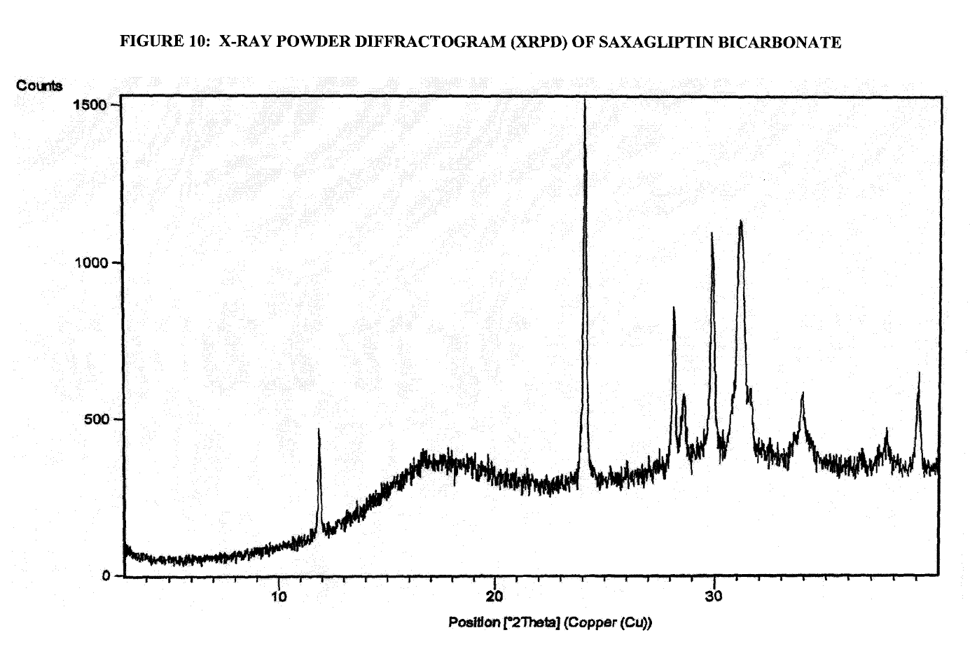

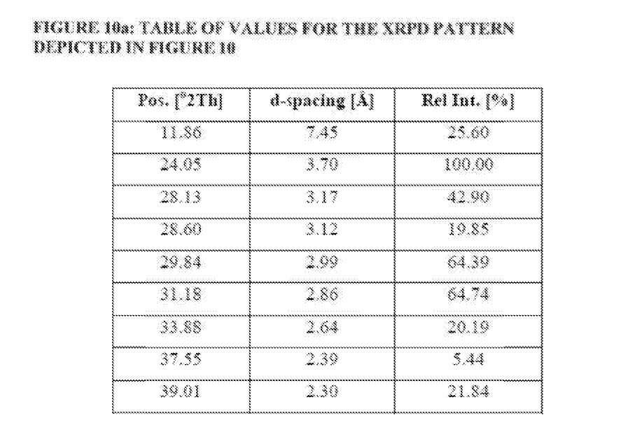

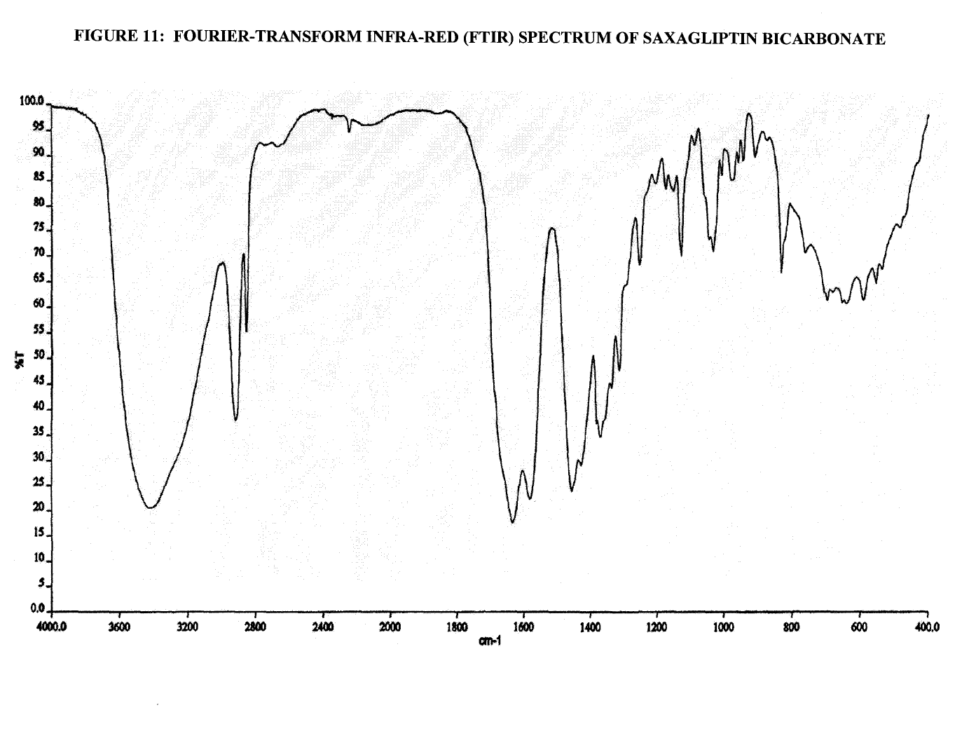

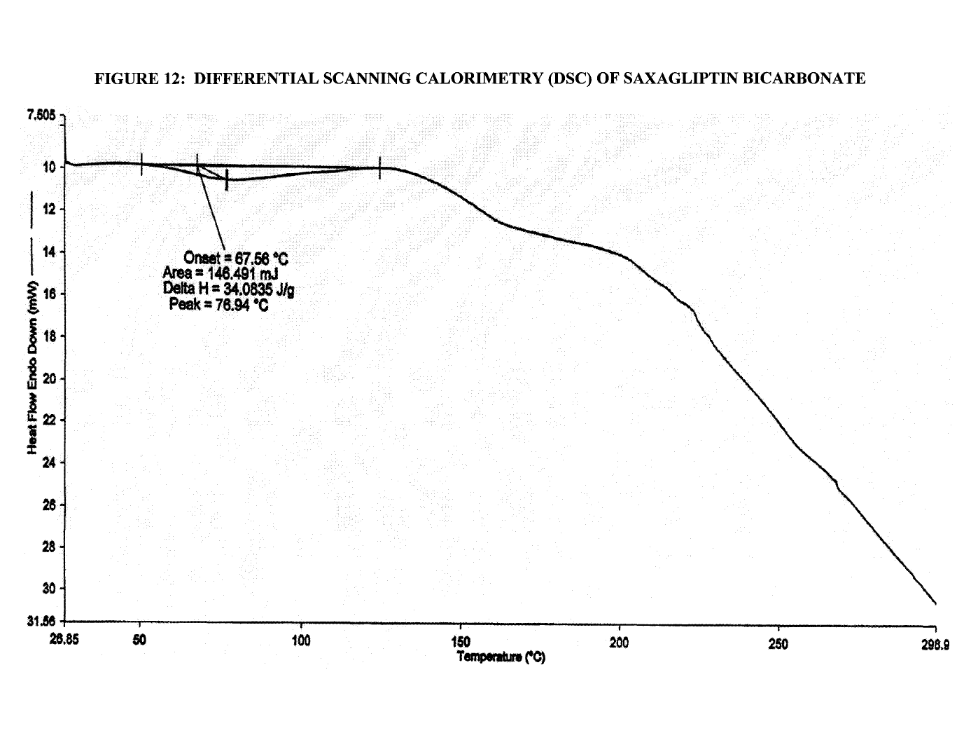

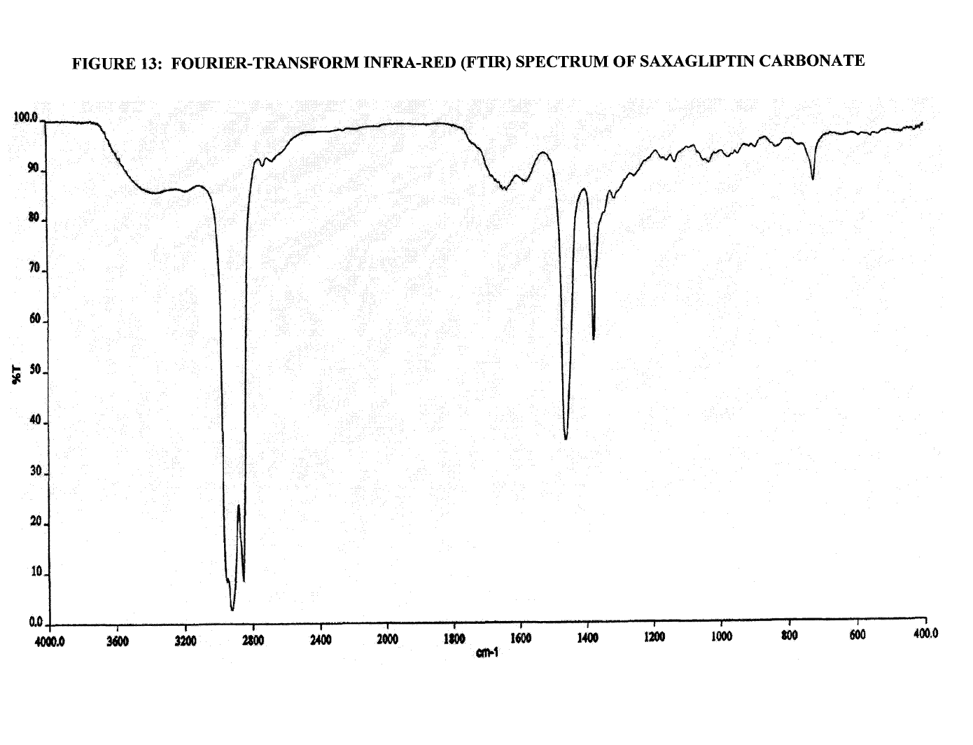

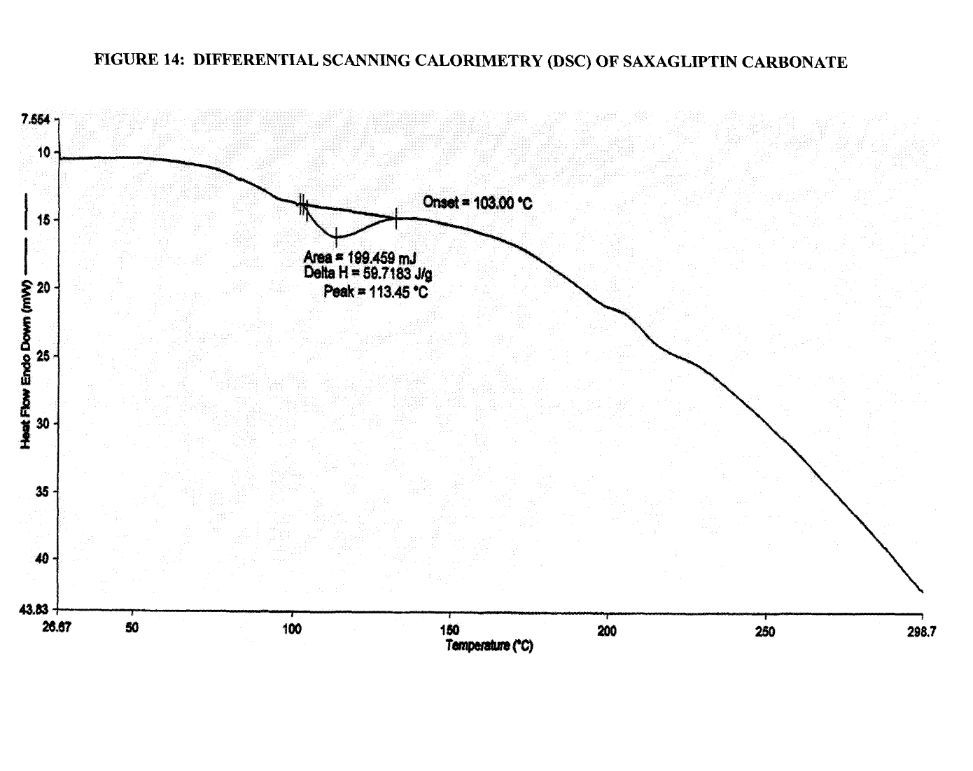

The XRPD of the samples were determined by using Instrument: PANalytical; Mode: Expert PRO; Detector: Xcelerator; ScanRange: 3-40; Step size: 0.02; Range: 3-40 degree 2 theta; CuKα radiation at 45 kV.

FTIR of the samples was determined by using Instrument: Perkin Elmer; SCAN: 16 scans, Resolution: 4.0 cm⁻¹; potassium bromide pellet method.

DSC of the samples was determined by using Instrument: Perkin Elmer, Diamond DSC. Data collection parameters: Scanning rate: 10° C./min; Temperature: 30° C.-300° C.

While the present invention has been described in terms of its specific embodiments, certain modifications and equivalents will be apparent to those skilled in the art, and are intended to be included within the scope of the present invention.

### EXAMPLES

### Example 1

### Preparation of Saxagliptin Bisulphate

A mixture of saxagliptin (1.5 g) and ethyl acetate (30 mL) was stirred and cooled to −5° C., followed by drop-wise addition of sulphuric acid solution (0.466 g) in ethyl acetate (15 mL) at −5° C. to 0° C. for 5 minutes. The temperature of the reaction mixture was raised to 15° C. with continuous stirring for 15 minutes, followed by the addition of methanol (4.5 mL), and then stirred for 30 minutes at 15° C. to 20° C. The solvent was distilled off from the reaction mixture completely under vacuum at 35° C. Ethyl acetate (45 mL) was charged, and stirred at 20° C. to 22° C. for 14 hours to obtain the solid. The solid was filtered under vacuum in nitrogen environment, washed with n-hexane (20 mL), and dried under vacuum at 35° C. to 40° C. for about 7 hours to obtain the title compound.

### Yield: 1.5 g

### Example 2

### Preparation of Saxagliptin Acetate

A mixture of saxagliptin (1.13 g) and isopropanol (10 mL) was stirred, and cooled to 5° C., followed by the addition of acetic acid (0.22 g) at 5° C. to 10° C., and then stirred for 1 hour while gradually raising the temperature to about 20° C. to 25° C. The reaction mixture was cooled to 0° C. to 5° C., and then stirred for 4 hours, maintaining the same temperature, to obtain a white solid. The solid was filtered through a sintered funnel under vacuum in a nitrogen environment, washed with isopropanol (10 mL), and dried under vacuum at 40° C. to 45° C. for 12 hours to 14 hours to obtain the title compound.

### Yield: 400 mg

### Example 3

### Preparation of Saxagliptin Acetate from Saxagliptin Trifluoroacetate

A mixture of saxagliptin trifluoroacetate (1.5 g) and dichloromethane (30 mL) was charged in a round bottomed flask, stirred, and cooled to 0° C. 8% Aqueous sodium bicarbonate solution (9 mL) was added drop-wise to the reaction mixture over 5 minutes at 0° C. to 2° C. The mixture was stirred, and the temperature was raised to 18° C. Sodium chloride (4.5 g) was added, and stirred at 17° C. to 19° C. for 25 minutes, and filtered through a cotton plug. The filtrate obtained was allowed to settle for 10 minutes. The aqueous layer was extracted thrice with dichloromethane (10 mL). All the organic layers were combined. Sodium sulphate was added, and filtered through a cotton plug. The filtrate was cooled to 15° C. in 5 minutes. Dichloromethane-acetic acid solution (2 mL of dichloromethane in 0.2 mL of acetic acid) was added to the filtrate at 15° C. The solvent was distilled off to the maximum extent under reduced pressure at 15° C., and then ethyl acetate (15 mL) was added, and stirred at 15° C. for 30 minutes. The solid obtained was filtered under vacuum, washed with chilled ethyl acetate (5 mL), and kept under vacuum suction for 5 minutes. The material obtained was dried under vacuum at 20° C. to 25° C. for 14 hours to 16 hours to obtain the title compound.

### Yield: 840 mg

### Example 4

### Preparation of Saxagliptin Oxalate

A mixture of saxagliptin (1.0 g) and dichloromethane (15 mL) was stirred and cooled to 0° C., followed by the addition of oxalic acid (0.358 g) and isopropanol (3 mL) at 0° C. The reaction mixture was stirred for 2 hours at 0° C. to 5° C., and further stirred for another 2 hours at 8° C. to 10° C. to obtain the white solid. The solid was filtered under vacuum, washed with dichloromethane (5 mL), and dried under vacuum at 33° C. to 37° C. for 8 hours to obtain the title compound.

### Yield: 200 mg

### Example 5

### Preparation of Saxagliptin Bicarbonate

Saxagliptin hydrochloride (5 g) was dissolved in water (50 mL) at 22° C., stirred, and cooled to 1° C. in 15 minutes, followed by drop-wise addition of 10% aqueous potassium carbonate solution (20 mL) at 0° C. to 5° C. over 10 minutes to adjust the pH to 8.3. The reaction mixture was stirred for 10 minutes at 5° C. and the pH was checked and adjusted to 8.3. The water in the reaction mixture was distilled off under vacuum at 25° C. in 2 hours, followed by the addition of ethanol (60 mL). The inorganic substances in the reaction mixture were filtered under vacuum. The inorganic substances were washed with ethanol (15 mL) and filtered to obtain filtrate. The obtained filtrates were distilled off under vacuum at 25° C. in 1 hour to obtain an oily mass.

The oily mass of saxagliptin free base (6.2 g) was dissolved in ethanol (15 mL) at 25° C., and stirred for 10 minutes. Dry ice (50 g) was added slowly under stirring in 35 minutes. Ethanol was distilled off under vacuum completely at 25° C. in 30 minutes to obtain a sticky mass. Ethyl acetate (60 mL) was added to the sticky mass, and stirred at 25° C. for 30 minutes to obtain a solid. The solid obtained was filtered, washed with ethyl acetate (10 mL), and then dried under vacuum for 14 hours at 25° C. to obtain the title compound.

### Yield: 2.52 g

### Example 6

### Preparation of Saxagliptin Carbonate

Saxagliptin bicarbonate (300 mg) obtained as per Example 5 was dissolved in water (30 mL) and stirred for 4 hours at 65° C. to 70° C. The water in the reaction mixture was recovered at 65° C. to 70° C. under vacuum to obtain a residue. To the residue, ethyl acetate (20 mL) was added, stirred for 10 minutes at 55° C., and the solvent was recovered under vacuum at 50° C. to 55° C. to obtain a solid mass. To the solid mass, ethyl acetate (10 mL) was added, and stirred for 15 minutes. The solid obtained was filtered, then dried under vacuum at 65° C. for 14 hours to obtain the title compound.

Yield: 150 mg

1. Saxagliptin bisulphate.

2. The saxagliptin bisulphate of claim 1, in crystalline form.

3-10. (canceled)

11. Saxagliptin acetate.

12. The saxagliptin acetate of claim 11, in crystalline form.

13-20. (canceled)

21. Saxagliptin oxalate.

22. The saxagliptin oxalate of claim 21, in crystalline form.

23-30. (canceled)

31. A process for the preparation of a compound of Formula I 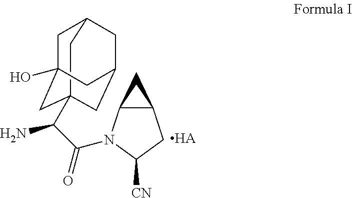
which comprises treating saxagliptin or its salt with HA, wherein HA is selected from sulphuric acid, acetic acid, or oxalic acid.

32-41. (canceled)

42. Saxagliptin bicarbonate of Formula II.

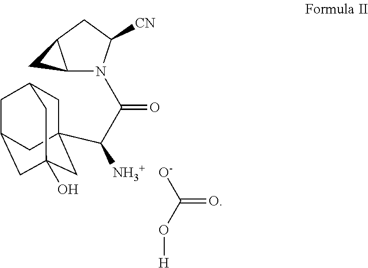

43. The saxagliptin bicarbonate of claim 42, in crystalline form.

44-51. (canceled)

52. A process for the preparation of saxagliptin bicarbonate, which comprises contacting saxagliptin or its salt with a suitable carbonate source.

53-59. (canceled)

60. Saxagliptin carbonate of Formula III.

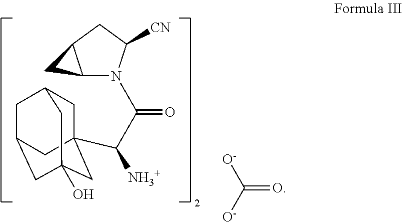

61. The saxagliptin carbonate of claim 60, in crystalline form.

62-64. (canceled)

65. A process for the preparation of saxagliptin carbonate, which comprises heating saxagliptin bicarbonate in the presence of water.

66-72. (canceled)
